(19) United States
(12) Patent Application Publication
(10) Pub. No.: US 2023/0106719 A1
(43) Pub. Date: Apr. 6, 2023

(54) DISSOLVING AGENT

(71) Applicants: NATIONAL UNIVERSITY CORPORATION HOKKAIDO UNIVERSITY, Hokkaido (JP); KAWASAKI INSTITUTE OF INDUSTRIAL PROMOTION, Kanagawa (JP)

(72) Inventors: Hideyoshi Harashima, Sapporo-shi (JP); Yusuke Sato, Sapporo-shi (JP); Saed Amjad Yousef Abbasi, Sapporo-shi (JP); Shinji Yamashita, Otsu-shi (JP); Haruki Higashino, Kashihara-shi (JP)

(21) Appl. No.: 17/760,269

(22) PCT Filed: Feb. 12, 2021
(86) PCT No.: PCT/JP2021/005310

§ 371 Date: Aug. 5, 2022

(30) Foreign Application Priority Data
Feb. 12, 2020 (JP) 2020-021811

(51) Int. Cl. A61K 9/08; A61K 47/14

(52) U.S. Cl. CPC A61K 9/08; A61K 47/14

(57) ABSTRACT

The present invention addresses the problem of providing a dissolving agent which improves the solubility of a compound such as curcumin that contains an aromatic ring without changing the structure of this compound. The present invention is a compound which is represented by general formula (1). (In the formula, R¹ is an alkyl group having 7 to 24 carbon atoms or an alkenyl group; A¹ represents an optionally substituted aryl group; Z¹ represents a single bond, an alkylene group having from 1 to 6 carbon atoms, or an alkenylene group having from 2 to 5 carbon atoms; and Z² represents a divalent linking group.)

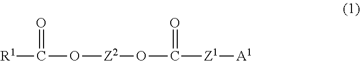

### TECHNICAL FIELD

The present invention relates to a compound useful as a dissolving agent of a poorly water-soluble compound having an aromatic ring, and a dissolving agent composed of this compound.

Priority is claimed on Japanese Patent Application No. 2020-021811, filed Feb. 12, 2020, the content of which is incorporated herein by reference.

### BACKGROUND ART

Intravenous (iv) injection is a main route of administration for a wide range of therapeutic agents, especially anticancer agents, because it tends to maximize the distribution of drugs in the tumor tissue compared to other routes of administration. In the case of formulating poorly water-soluble drugs into injectables, the drugs are generally dissolved in an oils or lipids and then dispersed in water to prepare a lipid-based formulation. The main lipid dispersion-based formulations include those in which a medicinal ingredient is loaded in liposome nanoparticles (NPs) and those in the form of an emulsion.

Iv-administered drug-loaded lipid nanoparticles accumulate in tumors due to passive targeting by the enhanced permeability and retention (EPR) effect or active targeting by interaction with specific receptors on tumor endothelium or cancer cells. In order to obtain a high therapeutic effect, it is important that more drug-loaded lipid nanoparticles are accumulated in the target tissue. Further, in order for more lipid nanoparticles to be accumulated in the target tissue, it is necessary for the lipid nanoparticles to circulate in the body for a sufficiently long time. The blood concentration-time profile of lipid nanoparticles is an important parameter that determines the ability to accumulate in tumors.

Unfortunately, it has been widely reported that drugs leak rapidly out of the lipid nanoparticles that have entered the systemic circulation (see, for example, Non-Patent Document 1). This phenomenon is called premature drug release. Premature drug release is one of the major obstacles for drug delivery, since the drug is lost in the blood before it reaches the target tissue, for example, a tumor.

For example, curcumin, which is a low molecular weight hydrophobic natural compound, has various pharmacological effects such as anticancer effects and anti-inflammatory effects, and is also highly safe (see, for example, Non-Patent Document 2 or 3). For this reason, curcumin is expected to be applied as an active ingredient of pharmaceutical products. Similar to other poorly water-soluble compounds, curcumin can be formed into a lipid-based formulation, which is obtained by dispersing curcumin dissolved in an oil-based dissolving agent into lipid nanoparticles and dispersing them in water.

### CITATION LIST

Non-Patent Documents
Non-Patent Document 1: Rodzinski et al., Scientific Reports, 2016, 6:20867.
Non-Patent Document 2: Anand et al., Cancer Letters, 2008, vol. 267 (1), p. 133-164.
Non-Patent Document 3: Pavan et al., Nutrients, 2016, 8, 628; doi: 10.3390/nu8110628.
Non-Patent Document 4: Abbasi et al., International Journal of Nanomedicine, 2016, vol. 11, p. 2685-2694.
Non-Patent Document 5: Chao et al., Molecules, 2018, vol. 23 (7), 1568.

### SUMMARY OF INVENTION

### Technical Problem

Curcumin has low solubility in water and is easily degraded. For this reason, the lipid dispersion-based formulation of curcumin has various problems, such as low bioabsorption. Conventionally, there is a technique such as PEGylation as a technique for improving solubility, but for a low molecular weight compound containing an aromatic ring such as curcumin, a technique for not only improving water solubility but also improving absorption in an animal body has not been well known so far.

An object of the present invention is to provide a dissolving agent that improves the solubility of a compound containing an aromatic ring such as curcumin without changing the structure of this compound.

### Solution to Problem

The inventors of the present invention have found that an ester of monoolein and trans-cinnamic acid has a high affinity against a compound containing an aromatic ring such as curcumin, and that this ester is useful as a dissolving agent of the compound containing an aromatic ring, thereby completing the present invention.

That is, the present invention provides the following compound, dissolving agent, and pharmaceutical composition.

[1] A compound of the following general formula (1):

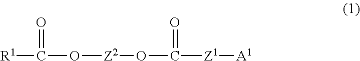

(in the formula, R¹ is an alkyl group or alkenyl group having 7 to 24 carbon atoms; A¹ is an aryl group which may have a substituent; Z¹ is a single bond, an alkylene group having 1 to 6 carbon atoms, or an alkenylene group having 2 to 6 carbon atoms; and Z² is a divalent linking group).

[2] The compound according to the above [1], which is of the following general formula (2-1) or (2-2):

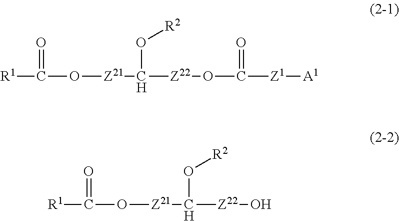

(in the formula, R¹, Z¹, and A¹ are defined in the formula (1); Z²¹ and Z²² are each independently a single bond, an alkylene group having 1 to 3 carbon atoms, or an alkenylene group having 2 to 3 carbon atoms; R² is a hydrogen atom, —CO—R¹, —NH—R¹, —CO—Z¹-A¹, or —NH—Z¹-A¹, and when a plurality of R¹, Z¹, or A¹ is present, the plurality of Z¹, or A¹ may be identical to or different from each other).

[3] The compound according to the above [1] or [2], which is of the following general formula (3-1), (3-2), or (3-3):

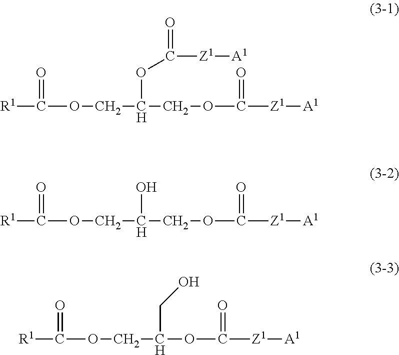

(in the formula, R¹, Z¹, and A¹ are defined in the formula (1), and in the formula (3-1), two of Z¹ and A¹ may be identical to or different from each other).

[4] The compound according to the above [3], wherein in the aforementioned general formulae (3-1), (3-2), and (3-3), R¹—COO— is an oleic acid residue or a palmitic acid residue,

Z¹ is a methylene group, an ethylene group, or a vinylene group, and

A¹ is a phenyl group which may have a substituent.

[5] The compound according to any one of the above [1] to [4], which is one or more selected from the group consisting of monoolein dicinnamate, monoolein monocinnamate, monopalmitin dicinnamate, monopalmitin monocinnamate, monoolein diphenyl butyrate, and monoolein monophenyl butyrate.

[6] A dissolving agent containing the compound of any one of the above [1] to [5].

[7] The dissolving agent according to the above [6], which is used to dissolve a compound having an aromatic ring.

[8] A pharmaceutical composition containing the compound of any one of the above [1] to [5].

[9] The pharmaceutical composition according to the above [8], further containing a compound having an aromatic ring.

### Advantageous Effects of Invention

Since the compound according to the present invention has an aromatic ring, it has a high affinity against a compound containing an aromatic ring and is useful as a dissolving agent of the compound containing an aromatic ring. By using the compound according to the present invention as a dissolving agent of a medicinal material containing an aromatic ring such as curcumin, a lipid dispersion-based formulation having favorable blood retention can be prepared.

### BRIEF DESCRIPTION OF DRAWINGS

FIG. 1 is a diagram showing a measurement result over time of relative amounts (%) with respect to the initial dose (ID) of DiD and curcumin in blood (% ID/mL (blood)) of ICR mice intravenously injected with curcumin-loaded NE (T80_coco_NE) into the tail vein in Example 2.

FIG. 2 is a diagram showing a measurement result of the solubility (mg/mL) of curcumin in coconut oil and CAOM in Example 3.

FIG. 3 is a diagram showing measurement results over time of relative amounts (%) with respect to the initial dose (ID) of curcumin in plasma (% ID/mL (plasma)) of ICR mice intravenously injected with each curcumin-loaded NE into the tail vein in Example 3.

FIG. 4 is a diagram obtained by plotting the Flory-Huggins interaction parameter (X_{ds}) of NE cores and curcumin with respect to the AUC of plasma profiles of curcumin of ICR mice intravenously injected with each curcumin-loaded NE into the tail vein in Example 3.

FIG. 5 is a diagram showing a measurement result of the solubilities (mg/mL) of quercetin (A), silicon phthalocyanine dihydroxide (B), and paclitaxel (C) in coconut oil and CAOM; and a measurement result (D) over time of relative amounts (%) with respect to the initial dose (ID) of paclitaxel in plasma (% ID/mL (plasma)) of ICR mice intravenously injected with paclitaxel-loaded NE into the tail vein in Example 4.

FIG. 6 is a diagram showing a measurement result over time of the concentration (mg/mL) of fenofibrate dissolved in a model small intestine solution from each fenofibrate-loaded NE formulation in Example 5.

FIG. 7 is a diagram showing a measurement result of the amount of a low-membrane-permeable drug migrated to the basal side by adding 0.1 to 100 μM CAOM LC and the low-membrane-permeable drug (atenolol (A), FD-4 (B), or Fexofenadine (C)) to the apical side of MDR-MDCKII cells planarly cultured on transwells in Example 5.

FIG. 8 is a diagram showing a measurement result of the solubility (mg/mL) of curcumin in a mixture of coconut oil and monopalmitin cinnamate in Example 6.

FIG. 9 is a diagram showing a measurement result of examining the solubility (mg/mL) of curcumin in a mixture of coconut oil and monoolein phenyl butyrate in Example 6.

### DESCRIPTION OF EMBODIMENTS

Hereinafter, embodiments of the present invention will be specifically described.

In the specification of the present application, a range of “X1 to X2 (X1 and X2 are real numbers satisfying a relationship of X1<X2)” means “X1 or more and X2 or less”.

Further, in the specification of the present application, a “compound of formula (X3)” may be expressed as a “compound (X3)”.

A compound according to the present invention is a compound of the following general formula (1).

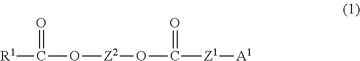

In the general formula (1), R¹ is an alkyl group or alkenyl group having 7 to 24 carbon atoms. The alkyl group or alkenyl group may be linear or branched. Examples of the alkyl group having 7 to 24 carbon atoms include a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an icosyl group, an eicosyl group, a henicosyl group, a heneicosyl group, a docosyl group, a tricosyl group and a tetracosyl group. Examples of the alkenyl group having 7 to 24 carbon atoms include a heptenyl group, an octenyl group, a nonenyl group, a decenyl group, an undecenyl group, a dodecenyl group, a tetradecenyl group, a pentadecenyl group, a hexadecenyl group, a heptadecenyl group, an octadecenyl group, a nonadecenyl group, an icosenyl group, an eicosenyl group, a henicosenyl group, a heneicosenyl group, a docosenyl group and a tricosenyl group. In the compound (1), R¹—COO— is preferably a fatty acid residue derived from a fatty acid (a group obtained by removing a hydrogen atom of a carboxylic acid group from the fatty acid) such as caprylic acid (octanoic acid), capric acid (decanoic acid), lauric acid (dodecanoic acid), myristic acid (tetradecanoic acid), pentadecylic acid (pentadecanoic acid), palmitic acid (hexadecanoic acid), margaric acid (heptadecanoic acid), stearic acid (octadecanoic acid), oleic acid (cis-9-octadecenoic acid), 11-octadecenoic acid, linolic acid (cis,cis-9,12-octadecadienoic acid), linolenic acid (octadecantrienoic acid), octadecatrienoic acid, eicosadienoic acid, eicosatrienoic acid, arachidonic acid (eicosatetraenoic acid), eicosanoic acid, behenic acid (docosanoic acid), lignoceric acid (tetracosanoic acid) or nervonic acid (cis-15-tetracosanoic acid). Among them, in the compound (1), R¹—COO— is preferably a caprylic acid residue, a capric acid residue, a lauric acid residue, a myristic acid residue, a palmitic acid residue, an oleic acid residue, a linoleic acid residue, a linolenic acid residue and an arachidonic acid residue, and is particularly preferably an oleic acid residue or palmitic acid residue having low toxicity to animals, especially humans.

In the general formula (1), A¹ is an aryl group which may have a substituent. Examples of the aryl group include a phenyl group, a naphthyl group, an anthryl group, a 9-fluorenyl group and an azulenyl group, and a phenyl group is particularly preferred.

The “aryl group which may have a substituent” is a group in which one or more hydrogen atoms, preferably 1 to 3 hydrogen atoms, bonded to a carbon atom of the aryl group are substituted with other functional groups. When two or more substituents are included, the substituents may be identical to or different from each other. Examples of the substituent include an alkyl group having 1 to 6 carbon atoms, an alkoxy group having 1 to 6 carbon atoms, and a hydroxy group. Examples of the alkyl group having 1 to 6 carbon atoms include a methyl group, an ethyl group, a propyl group, an isopropyl group, a butyl group, an isobutyl group, a sec-butyl group, a tert-butyl group, a pentyl group, an isopentyl group, a neopentyl group, a tert-pentyl group and a hexyl group. Examples of the alkoxy group having 1 to 6 carbon atoms include a methoxy group, an ethoxy group, a propoxy group, a butoxy group, a tert-butoxy group, a pentyloxy group and a hexyloxy group.

In the general formula (1), Z¹ is a single bond, an alkylene group having 1 to 6 carbon atoms, or an alkenylene group having 2 to 6 carbon atoms. The alkylene group having 1 to 6 carbon atoms may be linear or branched. Examples of the alkylene group having 1 to 6 carbon atoms include a methylene group, an ethylene group, a methylmethylene group, a trimethylene group, a dimethylmethylene group, a tetramethylene group, a pentamethylene group and a hexamethylene group. The alkenylene group having 2 to 6 carbon atoms may be linear or branched. Examples of the alkenylene group having 2 to 6 carbon atoms include a vinylene group, a 1-methylvinylene group, a propenylene group, a 1-butenylene group, a 2-butenylene group, a 1-pentenylene group and a 2-pentenylene group.

In the general formula (1), Z² is a divalent linking group. Examples of the divalent linking group include an alkylene group which may have a substituent, an alkenylene group which may have a substituent, —O—, —CO—, —COO—, —O—CO—, —NHCO—, —CONH—, and a divalent group in which these groups are appropriately combined. When Z² is an alkylene group which may have a substituent or an alkenylene group which may have a substituent, examples of the substituent which the alkylene group or the alkenylene group may have include —O—CO—Z¹-A¹, a fatty acid residue, an alkoxy group having 1 to 6 carbon atoms, and a hydroxy group.

Specific examples of the compound (1) include a compound (2-1) and a compound (2-2).

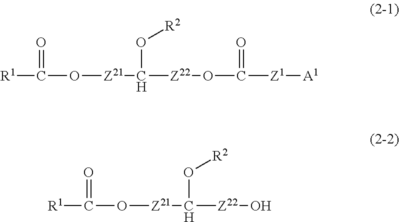

In the general formulae (2-1) and (2-2), R¹, Z¹ and A¹ are the same as those defined in the formula (1).

In the general formulae (2-1) and (2-2), R² is a hydrogen atom, —CO—R′, —NH—R′, —CO—Z¹-A¹, or —NH—Z¹-A¹. R¹, Z¹, and A¹ in R² are the same as those defined in the formula (1).

When there is a plurality of R′, Z¹, or A¹ in the general formulae (2-1) and (2-2), the plurality of R¹, Z¹, or A¹ may be identical to or different from each other. That is, when R² is —CO—R′ or —NH—R′, R¹ in R² may be identical to or different from R¹ ester-bonded to Z²¹ in the general formulae (2-1) and (2-2). When R² is —CO—Z¹-A¹ or —NH—Z¹-A¹, -Z¹-A¹ in R² may be identical to or different from -Z¹-A¹ ester-bonded to Z²² in the general formulae (2-1) and (2-2).

In the general formulae (2-1) and (2-2), Z²¹ and Z²² are each independently a single bond, an alkylene group having 1 to 3 carbon atoms, or an alkenylene group having 2 to 3 carbon atoms. Examples of the alkylene group having 1 to 3 carbon atoms include a methylene group, an ethylene group, a methylmethylene group, a trimethylene group and a dimethylmethylene group. Examples of the alkenylene group having 2 to 3 carbon atoms include a vinylene group, a 1-methylvinylene group, and a propenylene group.

The compound (2-1) and the compound (2-2) are preferably compounds in which R¹—COO— is a caprylic acid residue, a capric acid residue, a lauric acid residue, a myristic acid residue, a pentadecylic acid residue, a palmitic acid residue, a margaric acid residue, a stearic acid residue, an oleic acid residue, a 11-octadecenoic acid residue, a linoleic acid residue, a linolenic acid residue, an octadecatrienoic acid residue, an eicosadienoic acid residue, an eicosatrienoic acid residue, an arachidonic acid residue, an eicosanoic acid residue, a behenic acid residue, a lignoceric acid residue or a nervonic acid residue, Z¹ is an alkylene group having 1 to 3 carbon atoms or an alkenylene group having 2 to 3 carbon atoms, A¹ is a phenyl group which may have a substituent, R² is —CO—Z¹-A¹ or —NH—Z¹-A¹, Z^{2′} and Z²² are each independently a methylene group or an ethylene group; and are more preferably compounds in which R¹—COO— is a caprylic acid residue, a capric acid residue, a lauric acid residue, a myristic acid residue, a palmitic acid residue, an oleic acid residue, a linoleic acid residue, a linolenic acid residue or an arachidonic acid residue, Z¹ is a methylene group, an ethylene group, a trimethylene group, a vinylene group or a propenylene group, A¹ is a phenyl group which may have a substituent, R² is —CO—Z¹-A¹ or —NH—Z¹-A¹, and Z²¹ and Z²² are each independently a methylene group or an ethylene group.

As the compound (1), a compound (3-1), a compound (3-2) or a compound (3-3) is particularly preferred. In the general formulae (3-1), (3-2) and (3-3), R′, Z¹ and A¹ are the same as those defined in the formula (1). In the compound (3-1), two of Z¹ and A¹ in one molecule may be identical to or different from each other. The compound (3-1), the compound (3-2) and the compound (3-3) have a glycerol backbone or a structure similar thereto, and are likely to form micelles and lipid nanoparticles together with glycerophospholipids and the like that are generally used in producing a lipid dispersion-5 based formulation.

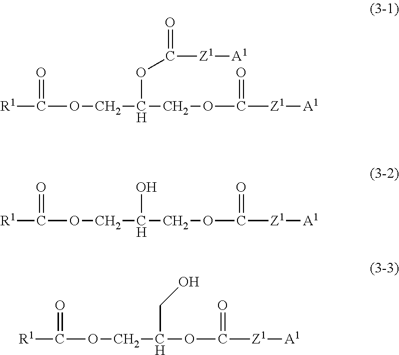

The compound (3-1), the compound (3-2) and the compound (3-3) are preferably compounds in which R¹—COO— is a caprylic acid residue, a capric acid residue, a lauric acid residue, a myristic acid residue, a pentadecylic acid residue, a palmitic acid residue, a margaric acid residue, a stearic acid residue, an oleic acid residue, a 11-octadecenoic acid residue, a linoleic acid residue, a linolenic acid residue, an octadecatrienoic acid residue, an eicosadienoic acid residue, an eicosatrienoic acid residue, an arachidonic acid residue, an eicosanoic acid residue, a behenic acid residue, a lignoceric acid residue or a nervonic acid residue, Z¹ is an alkylene group having 1 to 3 carbon atoms or an alkenylene group having 2 to 3 carbon atoms, and A¹ is a phenyl group which may have a substituent; are more preferably compounds in which R¹—COO— is a caprylic acid residue, a capric acid residue, a lauric acid residue, a myristic acid residue, a palmitic acid residue, an oleic acid residue, a linoleic acid residue, a linolenic acid residue or an arachidonic acid residue, Z¹ is a methylene group, an ethylene group, a trimethylene group, a vinylene group or a propenylene group, and A¹ is a phenyl group which may have a substituent; and are particularly preferably compounds in which R¹⁻—COO— is an oleic acid residue or a palmitic acid residue, Z¹ is a methylene group, an ethylene group or a vinylene group, and A¹ is a phenyl group which may have a substituent.

The compound (1) can be synthesized by an esterification reaction between a fatty acid ester having a hydroxy group (R¹—COO— Z²—OH) and an aromatic carboxylic acid compound (A¹-Z¹—COOH). In addition, the compound (3-1), the compound (3-2) and the compound (3-3) can be synthesized by the following esterification reaction. These esterification reactions can be carried out by a conventional method.

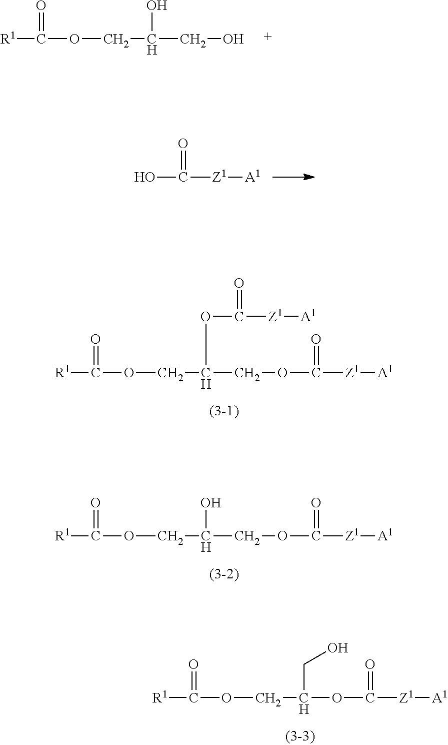

The compound (1) has an aromatic ring (A¹), and therefore has a high affinity against a compound having an aromatic ring, and is useful as a dissolving agent that dissolves the compound having an aromatic ring. The reason why the compound having an aromatic ring is highly soluble is not clear, but it is presumed that this is because a 7E-7E interaction acts between the aromatic ring (A¹) in the compound (1) and the aromatic ring in the compound to be dissolved.

Furthermore, the compound (1) has a hydrophobic hydrocarbon chain R¹⁻ in addition to the aromatic ring (A¹), and is highly lipophilic. Therefore, the compound (1) is also dissolved well in an oil medium such as vegetable oil, and is stably present in an oil core (internal fat-soluble portion) of emulsion particles in an oil-in-water emulsion. In addition, the affinity against the constituent lipids of lipid nanoparticles such as micelles and liposomes is also high, and the compound (1) is retained inside these lipid membranes.

As described above, the compound (1) has a high affinity against the compound having an aromatic ring, has favorable solubility in an oil medium, and can be stably present inside the oil core of the emulsion and the lipid membrane of the lipid nanoparticles. Therefore, the compound (1) is suitable as a dissolving agent of both an aqueous medium and an oil medium of the compound having an aromatic ring. In particular, the compound (1) is suitable as a dissolving agent used in producing a lipid dispersion-based formulation containing a compound having an aromatic ring as an active ingredient, since the compound having an aromatic ring can be relatively stably present inside the oil core of the emulsion or the lipid membrane of the lipid nanoparticles by the interaction with the compound (1).

Although the compound having an aromatic ring to be dissolved by the compound (1) (hereinafter, may be referred to as a “material to be solubilized”) is not particularly limited as long as it has an aromatic ring, since the 7E-7E interaction with the aromatic ring (A¹) in the compound (1) is likely to function, a low molecular weight compound is preferred, and a poorly water-soluble low molecular weight compound is more preferred. It should be noted that the term “poorly water-soluble compound” means a compound which requires at least 30 mL of water to dissolve 1 g of this compound as a solute.

Examples of the material to be solubilized include flavonoids, such as flavonols such as quercetin (CAS No: 117-39-5), flavanone, flavone, isoflavone, catechin and anthocyanidin; polyphenols other than flavonoids, such as curcumin (CAS No: 458-37-7), gingerol, resveratrol, tannin, and procyanidin; taxanes having aromatic rings, such as paclitaxel (CAS No: 33069-62-4) and docetaxel (CAS No: 114977-28-5); fenofibrate (CAS No: 49562-28-9); anthraquinones such as mitoxantrone dihydrochloride (CAS No: 70476-82-3); tetracycline-based antibiotics; and phthalocyanines such as silicon dihydroxyl phthalocyanine (CAS No: 19333-15-4).

The dosage form of the pharmaceutical composition produced by using the compound (1) as a dissolving agent is not particularly limited, and examples thereof include an oral agent, an injection, a suppository, an ointment, and a patch. Among them, a lipid dispersion-based formulation such as an injection is preferred because the effect of improving the solubility of the compound having an aromatic ring which is included in the compound (1) is more likely to be exhibited. The compound (1) is also preferable as a dissolving agent of a suppository, an ointment or a patch that uses an oily base material.

These formulations can be blended with a pharmaceutically acceptable carrier as necessary, and can be produced by a conventional preparation method known to those skilled in the art. As the pharmaceutically acceptable carrier, excipients, binders, disintegrants, lubricants and colorants in solid formulations; solvents, dissolving aids, suspending agents, isotonic agents, buffers and pain-relieving agents in liquid formulations, and the like are used. Further, if necessary, formulation additives such as preservatives, antioxidants, colorants, sweeteners and stabilizers may also be used.

In the case of producing a lipid dispersion-based formulation using the compound (1) as a dissolving agent, only one type of the compound (1) may be used, or two or more types of the compound (1) may be used in combination. Furthermore, various oily materials and amphipathic molecules other than the compound (1) may also 5 be used in combination. As the oily material, plant-derived oil such as coconut oil, synthetic oil and the like may be used. Further, as the amphipathic molecule, one or more lipids and surfactants generally used to form liposomes, such as phospholipids, sterols, and saturated or unsaturated fatty acids may be used in combination. More specifically, for example, the lipids described in International Patent Publication No. 2015/178343 may be appropriately used.

In the case of producing a lipid dispersion-based formulation, as the oily material or amphipathic molecule used in addition to the compound (1), it is preferable to select a material having a high affinity against the material to be solubilized. For example, when the material to be solubilized is a compound having a hydroxy group such as curcumin, it is preferable to use a surfactant having a group capable of forming a hydrogen bond with the hydroxy group, for example, a hydroxy group, an ethereal oxygen molecule, an amide bond or the like.

The lipid dispersion-based formulation in the form of an oil-in-water emulsion or the lipid-dispersion-based preparation in which lipid nanoparticles are dispersed in an aqueous medium can be produced by, for example, emulsifying a mixture obtained by mixing an oil medium composed of the compound (1) or a mixture of the compound (1) and other lipids, an aqueous medium, and a material to be solubilized by an emulsifier such as a homogenizer, an ultrasonic emulsifier, a high-pressure jet emulsifier or the like.

Further, it can also be produced by a well-known method to produce liposomes, for example, a reverse phase evaporation method or the like. If it is desired to control the size of lipid nanoparticles, an extrusion process (extrusion filtration) may be performed under high pressure using a membrane filter having a uniform pore diameter or the like.

The constitution of the aqueous solvent (dispersion medium) is not particularly limited, and examples thereof include a buffer solution such as a phosphate buffer solution, a citrate buffer solution and a phosphate buffered physiological saline solution, a physiological saline solution, and a cell culture medium. Although these aqueous solvents (dispersion media) can stably disperse lipid nanoparticles, monosaccharides such as glucose, galactose, mannose, fructose, inositol, ribose and xylose, disaccharides such as lactose, sucrose, cellobiose, trehalose and maltose, trisaccharides such as raffinose and melezitose, polysaccharides such as cyclodextrin, sugars (aqueous solutions) of sugar alcohols and the like such as erythritol, xylitol, sorbitol, mannitol and maltitol, and polyhydric alcohols (aqueous solutions) such as glycerin, diglycerin, polyglycerin, propylene glycol, polypropylene glycol, ethylene glycol, diethylene glycol, triethylene glycol, polyethylene glycol, ethylene glycol monoalkyl ether, diethylene glycol monoalkyl ether and 1,3-butylene glycol may be further added. In order to stably store the lipid nanoparticles dispersed in the aqueous solvent for a long period of time, it is desirable to eliminate the electrolyte in the aqueous solvent as much as possible from the viewpoint of physical stability in terms of suppression of aggregation and the like. Further, in terms of the chemical stability of lipids, it is desirable to set the pH of the aqueous solvent from weakly acidic to near neutral (pH of about 3.0 to 8.0), and/or to remove dissolved oxygen by nitrogen bubbling or the like.

The form of the lipid nanoparticles produced by using the compound (1) is not particularly limited, and for example, as a form dispersed in an aqueous solvent, a unilamellar liposome, a multilamellar liposome, a spherical micelle, an amorphous layered structure or the like can be mentioned.

The animal to which the pharmaceutical composition such as the lipid dispersion-based formulation produced by using the compound (1) is administered is not particularly limited, and may be a human or a non-human animal. Examples of the non-human animal include mammals such as cattle, pigs, horses, sheep, goats, monkeys, dogs, cats, rabbits, mice, rats, hamsters and guinea pigs, and birds such as chickens, quail and ducks. Further, the route of administration of the pharmaceutical composition to an animal is not particularly limited, but parenteral administration such as intravenous administration, enteral administration, intramuscular administration, subcutaneous administration, transdermal administration, nasal administration or pulmonary administration is preferred.

In the lipid dispersion-based formulation produced by using the compound (1), the material to be solubilized is stably retained in a hydrophobic portion of the oil-in-water emulsion or lipid nanoparticles due to the interaction with the compound (1). More specifically, in the oil-in-water emulsion, the material to be solubilized is retained together with the compound (1) inside the nanoemulsion particles (nanoparticles composed of oily components). In the lipid nanoparticles, the material to be solubilized is retained inside if the inside of the lipid nanoparticles is hydrophobic, and is retained in a lipid membrane constituting the lipid nanoparticles together with the compound (1) if the inside of the lipid nanoparticles is hydrophilic, respectively.

In the lipid dispersion-based formulation produced by using the compound (1), since the solubility of the material to be solubilized in the compound (1) improves and the material to be solubilized is stably retained together with the compound (1), when intravenously injected, the material to be solubilized is less likely to be released from the nanoemulsion particles and lipid nanoparticles in systemic circulation. As a result, in the lipid dispersion-based formulation produced by using the compound (1), the blood retention of the material to be solubilized improves, and the AUC (area under the blood concentration curve) also improves. As described above, in the present invention, the bioavailability of the material to be solubilized can be improved only by using the compound (1) as the dissolving agent without changing the structure of the material to be solubilized.

### EXAMPLES

Next, the present invention will be described in more detail with reference to Examples, but the present invention is not limited to the following Examples.

### <Reagent>

Curcumin was obtained from Wako Kagaku Co., Ltd. (Osaka, Japan). Tween 80, Pluronic F127, coconut oil, silicon phthalocyanine dihydroxide, and polypropylene glycol 1000 were purchased from Sigma-Aldrich. Paclitaxel was obtained from LC Laboratories and quercetin was obtained from Cayman Chemical. Trans-cinnamic acid (>98%), monoolein, triolein, N-ethyl-N′-(3-dimethylaminopropyl) carbodiimide hydrochloride (EDCI) and 4-dimethylaminopyridine (DMAP) were provided from Tokyo Chemical Industry Co., Ltd. Egg phosphatidylcholine (EPC) and 1,2-distearoyl-sn-glycero-3-phosphoethanolamine-N-[amino(polyethylene glycol)-2000] (DSPE-PEG2000) were purchased from NOF Corporation, and the Cell Counting Kit-8 was purchased from Dojindo Laboratories. The 1,1′-dioctadecyl-3,3,3′,3′-tetramethylindodicarbocyanine (DiD) fluorescent probe was supplied by Thermo Fisher Scientific.

### <Synthesis of CAOM and Determination of Drug Solubility>

The esterification of trans-cinnamic acid and glyceryl monooleate (monoolein) was carried out using a condensation reaction. Briefly, 1 equivalent of monoolein and 2.2 equivalents of trans-cinnamic acid were dissolved in dichloromethane. 2.5 equivalents of EDCI and 0.1 equivalents of DMAP were added thereto, and the resulting mixture was allowed to react overnight under continuous stirring. After evaporation of dichloromethane, the product was resuspended in ethyl acetate, and the resulting solution was filtered and washed twice with a saturated citric acid solution to remove unreacted basic species. The product was washed twice with a 0.1N sodium hydroxide solution in order to remove the unreacted trans-cinnamic acid. Finally, the product was dried using sodium sulfate, filtered and concentrated by evaporation.

In order to obtain purified monoolein dicinnamate or monoolein monocinnamate, monoolein (>70%) was passed through a silica column and the purified fraction of monoolein was recovered. The reaction with trans-cinnamic acid was carried out as described above. The purified monoolein dicinnamate or monoolein monocinnamate was recovered by column separation and confirmed by proton NMR.

Probe sonication was carried out for 20 seconds in order to measure the drug solubility in CAOM, and the mixture was shaded from light and allowed to stand for 2 days at room temperature for equilibration. Drug-dispersed CAOM was centrifuged at 15,000×g for 15 minutes to remove any undissolved drug powder. The concentrations of curcumin, quercetin, and silicon phthalocyanine dihydroxide were determined using a spectrophotometer (manufactured by Beckman Coulter) at spectral wavelengths of 434, 370, and 680 nm, respectively. The concentration of paclitaxel was determined using HPLC equipped with a C18 column (manufactured by Wako Kagaku Co., Ltd.). An isocratic mobile phase composed of acetonitrile and double distilled water (DDW) (55:45) was pumped at 1 mL/min, and the absorbance of paclitaxel was detected at 227 nm.

### <Preparation and Characterization of Curcumin-Loaded Nanoemulsions>

Nanoemulsions (NE) were prepared by the sonication method described in Non-Patent Document 4. For example, for the preparation of T80_coco_NE, a certain amount of curcumin, 55 mg of coconut oil and 40 mg of Tween 80 were mixed with 1 mL of an aqueous phase (HEPES buffer, 10 mM, pH 7.4) containing 5 mg of EPC and 5 mg of DSPE-PEG2000. For the preparation of F127_coco_NE, Pluronic F127 was first dissolved in the aqueous phase and added to the oil phase. In the case of CAOM cores, curcumin and 55 mg of CAOM were mixed with 1 mL of the aqueous phase containing 40 mg of nonionic surfactant, 5 mg of EPC, and 5 mg of DSPE-PEG2000. For the fluorescent labeling of NE, DiD was dissolved in the oil phase. After mixing all the components, the emulsion was sonicated for 2 to 3 minutes in an amplitude range of 20 to 25% using a probe-type sonifier (manufactured by Branson). The particle size, polydispersity index (PDI) and ζ potential were measured using a Malvern Zetasizer (manufactured by Malvern Instruments). After separating the unencapsulated curcumin powder from the NE dispersion, the encapsulation efficiency was estimated. NE was centrifuged at 10,000×g at room temperature for 10 minutes. The supernatant was discarded, and the curcumin pellet was washed with redistilled water (DDW) and further 5 centrifuged for 3 minutes. The obtained pellet was dissolved in dimethyl sulfoxide (DMSO), and the concentration of curcumin was quantified as described above. The encapsulation efficiency and drug loading (DL) were calculated using the following equations (F1) and (F2), respectively.

$$Encapsulation \% = 100 \% - ( \frac{conc . of curcumin pellet}{Total curcumin conc .} ⨯ 100 \% ) \qquad ( F1 )$$

$$DL = \frac{Wt . of curcumin}{Wt . of curcumin + Wt . of NE} ⨯ 100 \% \qquad ( F2 )$$

### <Evaluation of Curcumin Uptake and Cell Viability>

Human cervical cancer cell lines (Hela cells, 2×10⁵) were seeded in 6-well culture plates and incubated for 24 hours (37° C., 5% CO₂) in a serum-containing medium (+DMEM) before the treatment. The cells were incubated with 50 μM free curcumin (DMSO) or curcumin-loaded NE for 5 hours in +DMEM. After the end of the incubation period, the cells were washed 3 times with cold phosphate buffered saline (PBS), then trypsinized and collected by centrifugation (1,800×g, 3 minutes, 4° C.). The obtained pellet was washed once more with cold PBS, suspended in 0.5% bovine serum albumin containing PBS and 0.1% sodium azide, and analyzed using a FACS

Calibur flow cytometer (manufactured by BD Biosciences). The curcumin signal was detected by native green fluorescence using the FL1-H channel.

For the evaluation of cell viability, Hela cells were seeded in 96-well plates at a density of 0.5×10⁴ cells/well 24 hours prior to the treatment and cultured. 100 μM free curcumin (DMSO) or NE loaded with curcumin was added to each well, incubated for 5 hours, washed, and incubated with fresh medium for 19 hours. Subsequently, a cell viability assay (water-soluble tetrazolium-8 assay, WST-8) was carried out. The absorbance was measured using a microplate reader (PerkinElmer, Inc., Waltham, Mass.), and the relative cell viability was calculated as the ratio of absorbance of the treated wells with respect to that of the untreated ones.

### <Studies on Phase Transition>

100 μL of curcumin-loaded F127 or T80 micelle (5% w/v in HEPES buffer) was added to 900 μL of diisopropyl ether, and the resulting solution was continuously rotated at 4 rpm. 300 μL of diisopropyl ether was withdrawn at various time points from the micelle dilution, and replaced with an equal amount of fresh diisopropyl ether. The recovered sample was evaporated to dryness, the remaining curcumin was dissolved in DMSO, and its absorption was measured at λ=434 nm.

### <Measurement of Drug Concentration in Blood or Plasma>

All the animal experiments were reviewed and approved by the Hokkaido University Animal Committee.

ICR mice (female, 4 to 5 weeks old) were intravenously injected with drug- or DiD-loaded NEs (6 to 7 mg/kg curcumin or paclitaxel, or 0.32 mg/kg DiD). After a certain period of time, blood samples were taken from the tail vein for subsequent experiments. In the case of DiD-labeled NEs, 20 μL of blood was dissolved in 216 μL of 1% w/v sodium lauryl sulfate in order to evaluate fluorescence intensity at irradiation and excitation wavelengths of 624 and 669 nm, respectively (Infinite M200 Plate Reader, manufactured by Tecan).

In order to evaluate the curcumin concentration in blood, 20 μL of blood was mixed with 180 μL of DMSO: methanol (1:4) to extract curcumin and precipitate blood proteins. The mixture was centrifuged at 4° C. and 5,000 rpm for 20 minutes, and 20 μL of the supernatant was injected into an HPLC system (LaChrom, manufactured by Hitachi, Ltd.). The concentration of curcumin was measured at an absorption wavelength of 430 nm using a method modified from the method reported in Non-Patent Document 5. Briefly, an isocratic mobile phase composed of acetonitrile and 0.4% acetic acid (DDW solution) (48:52) was pumped to the Wakopak (registered trademark) Ultra C18-5 4.6 mm×150 mm column (manufactured by Wako Kagaku Co., Ltd.) at a flow rate of 1 mL/min. In order to evaluate the concentration of curcumin or paclitaxel in plasma, 40 μL of blood was collected and immediately centrifuged at 5,000 rpm and 4° C. for 10 minutes. 10 μL of plasma was mixed with 50 μL of tert-butyl alcohol and DMSO (4:1) and centrifuged to obtain a supernatant (14,000×g, 10 minutes, 4° C.). The drug concentration was evaluated using HPLC as described above. The area under the curve (AUC) of plasma profiles was calculated using the trapezoidal method.

### <Theoretical Calculation of Drug Interaction Affinity>

The Flory-Huggins interaction parameter (X_{ds}) of curcumin or paclitaxel with the NE cores was calculated using the modified equation by Beerbower. Here, A is the difference in solubility between the drug (d) and the solvent (s), δ_{d}, δₚ, and δₕ are the partial Hansen solubility parameters (HSPs) of the drug and the solvent, V_{d} is the molar volume of the drug, R is the gas constant, and T is the absolute temperature in Kelvin.

$$A = \sqrt{{( {δ}_{d , s} - {δ}_{d , d} )}^{2} + {( {δ}_{p , s} - {δ}_{p , d} )}^{2} + {( {δ}_{h , s} - {δ}_{h , d} )}^{2}} \qquad ( F3 )$$

$${X}_{d s} = \frac{{A}^{2} {V}_{d}}{R T} \qquad ( F4 )$$

The partial solubility parameters for each pure material were calculated by the group contribution method using the following equations (F5) to (F7). Since the NE cores were regarded as a solvent mixture of the oil phase and the surfactant tails, the HSP value of a certain NE core was calculated by multiplying the HSP of each pure material by the volume ratio in the core. F_{di} is the dispersion attraction constant, Fₚᵢ is the polar attraction constant, Eₕᵢ is the hydrogen bonding energy, and Vᵢ is the molar volume.

$${δ}_{d} = \frac{Σ {F}_{di}}{Σ {V}_{i}} \qquad ( F5 )$$

$${δ}_{p} = \frac{\sqrt{Σ {F}_{pi}^{2}}}{Σ {V}_{i}} \qquad ( F6 )$$

$${δ}_{h} = \sqrt{\frac{Σ {E}_{h i}}{Σ {V}_{i}}} \qquad ( F7 )$$

### <Plasma Biochemical Marker>

Following intravenous bolus injection of NEs into ICR mice (female, 4 to 5 weeks old), whole blood was collected from the inferior vena cava and centrifuged at 5,000 rpm and 4° C. for 10 minutes. Biochemical markers in plasma were measured using a JCA-BM6050 automated analyzer (manufactured by JEOL). Aspartate transaminase (AST), alanine transaminase (ALT), and lactate dehydrogenase (LDH) were measured using the method recommended by the Japan Society of Clinical Chemistry (JSCC). Blood urea nitrogen (BUN) was measured using the Urease-GLDH method, and creatinine (CRE) was measured using the creatinine amidohydrolase-creatinine amidinohydrolase-SOX-POD method.

### <Statistical Analysis>

The statistical significance between two groups was analyzed using a two-tailed Student's t-test of two unpaired independent groups. For multiple comparisons among three or more groups, non-repeated ANOVA followed by Bonferroni correlation were used, unless otherwise stated. Comparisons with untreated samples were made using Dunnett's test. A statistically significant difference was set at p<0.05.

### Example 1

Focusing on aromatic rings present in curcumin, attempts were made to improve the interaction between the fat-soluble core of the nanoemulsion and curcumin by utilizing the 7C-7C interactions between the aromatic rings.

As materials, monoolein and trans-cinnamic acid that were abundant in nature and considered to have extremely low toxicity to humans were used. By the esterification reaction of both materials, a mixture of monoolein dicinnamate and monoolein monocinnamate (about 2:1 in molar ratio, both of which were oil-like materials) was obtained. The mixture of monoolein dicinnamate and monoolein monocinnamate was named cinnamic acid-derived oil-like material (CAOM) and was used in subsequent experiments.

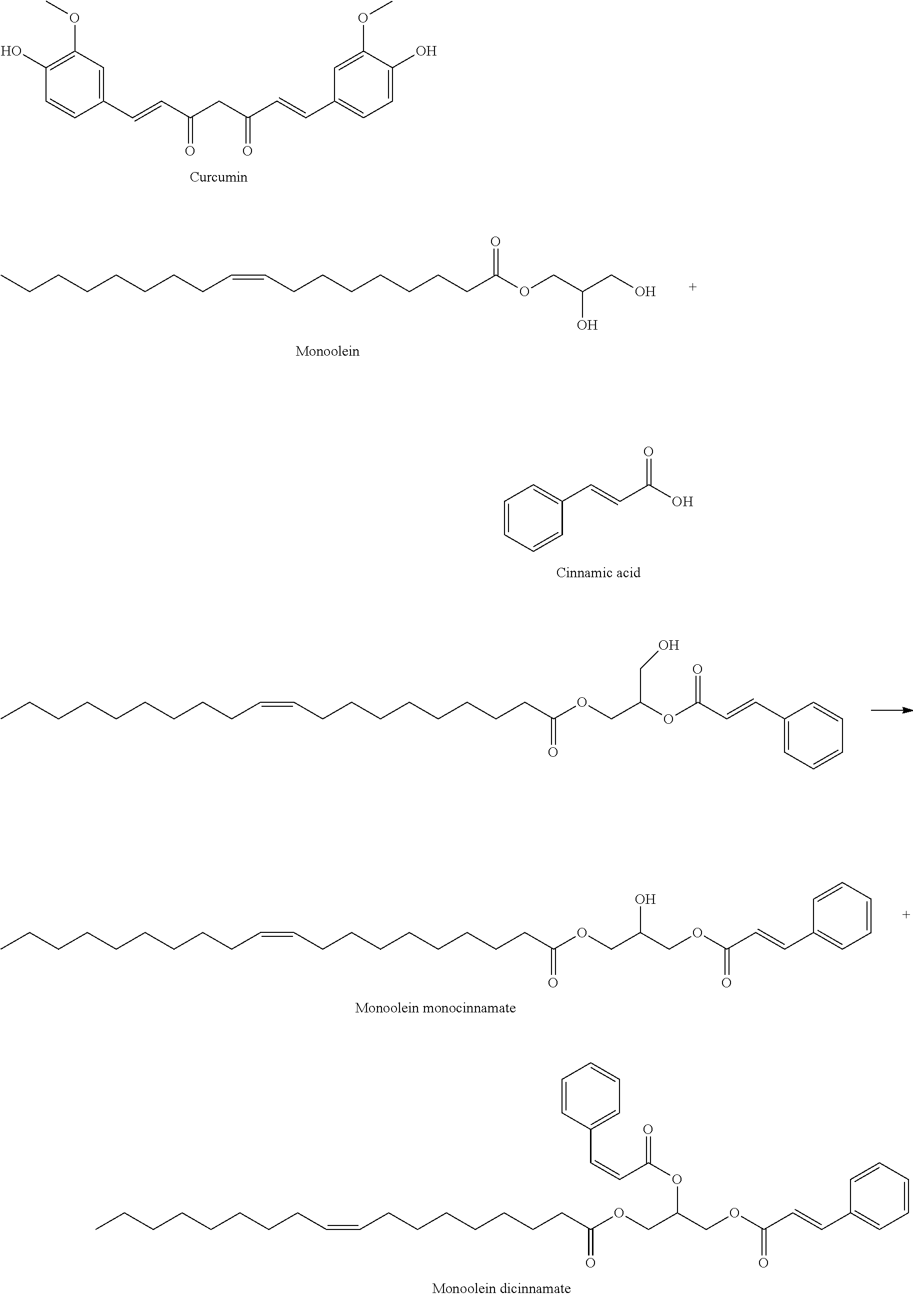 [SMILES: COc1cc(/C=C/C(=O)CC(=O)/C=C/c2ccc(O)c(OC)c2)ccc1O ; CCCCCCCC/C=C\CCCCCCCC(=O)OCC(O)CO ; O=C(O)/C=C/c1ccccc1 ; CCCCCCCCC/C=C\CCCCCCCCC(=O)OCC(CO)OC(=O)/C=C/c1ccccc1 ; CCCCCCCCC/C=C\CCCCCCCCC(=O)OCC(O)COC(=O)/C=C/c1ccccc1 ; CCCCCCCC/C=C\CCCCCCCC(=O)OCC(COC(=O)/C=C/c1ccccc1)OC(=O)/C=C\c1ccccc1]

### Example 2

The mode of release of curcumin from the nanoemulsion was examined.

A nanoemulsion (NE) composed of an oil phase and a nonionic surfactant as the main emulsifier was formulated and used to incorporate curcumin. Table 1 shows the constitution and characteristics of each NE. Each NE particle had an oily component (coconut oil or CAOM) as a core, and an amphipathic phospholipid (EPC and DSPE-PEG2000) and a surfactant (Tween 80 and Pluronic F127) were arranged on the surface thereof. The oil core also includes hydrophobic moieties of the phospholipid and surfactant. These formulations were labeled with DiD as an indicator of the hemodynamics of the NE particles themselves.

TABLE 1

| | T80_coco_NE | F127_coco_NE | T80_caom_NE | F127_caom_NE |
|---|---|---|---|---|
| Coconut oil (% w/v) | 5.5 | 5.5 | — | — |
| CAOM (% w/v) | — | — | 5.5 | 5.5 |
| Tween 80 (% w/v) | 4 | — | 4 | — |
| Pluronic F127 (% w/v) | — | 4 | — | 4 |
| EPC (% w/v) | 0.5 | 0.5 | 0.5 | 0.5 |
| DSPE-PEG2000 (% w/v) | 0.5 | 0.5 | 0.5 | 0.5 |
| DL (wt %) | 1.1 ± 0.04 | 1.0 ± 0.03 | 1.2 ± 0.13 | 1.2 ± 0.08 |
| Encapsulation % | 99.9 ± 0.07 | 92.9 ± 2.63 | 99.9 ± 0.052 | 99.8 ± 0.084 |
| Size (nm) | 71.2 ± 2 | 87.8 ± 29 | 45.1 ± 5.1 | 66.1 ± 3.9 |
| PDI | 0.23 ± 0.02 | 0.15 ± 0.01 | 0.13 ± 0.03 | 0.15 ± 0.05 |
| ξ-potential | −13.8 ± 1.5 | −4.0 ± 3.3 | −13.4 ± 2.3 | −5.7 ± 1.0 |

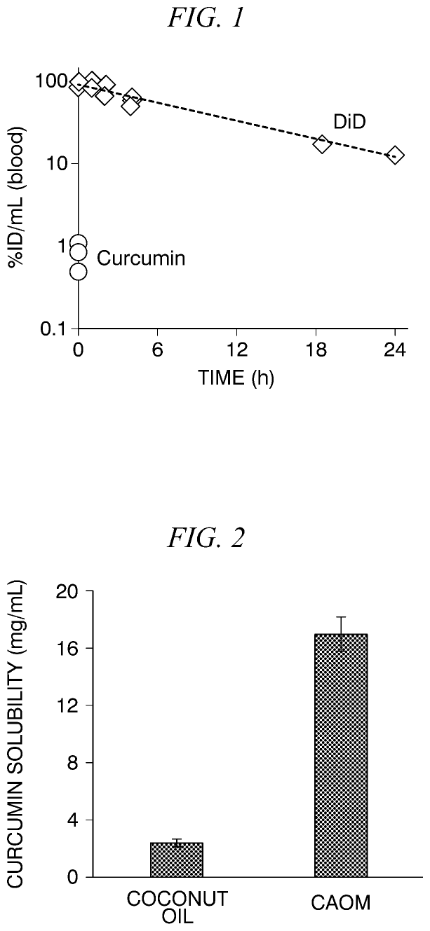

First, T80_coco_NE was injected into the tail vein of ICR mice, and the concentrations of DiD and curcumin in blood were measured over time. The concentration of curcumin was measured using HPLC and the concentration of DiD was determined by its fluorescence (n=3). The measurement results of the relative amount (%) with respect to the initial dose (ID) (mL) of DiD and curcumin in blood are shown in FIG. 1. Since the surface of the NE particles contained a high concentration of polyethylene glycol (PEG) imparted by the hydrophilic heads of Tween 80 and DSPE-PEG2000, a considerable amount of DiD (>90% ID/mL blood) remained in the blood 1 hour after the tail vein administration, but the amount of DiD decreased to about 10% after 24 hours.

On the other hand, curcumin in the blood was not detected from 1 minute after intravenous administration in the tail vein. Free curcumin in the blood is rapidly eliminated from the blood due to extensive metabolism in the liver and other organs. Therefore, the complete elimination of curcumin within 1 minute after intravenous administration of curcumin-loaded NE suggested that curcumin was prematurely released from the NE particles into the blood circulation and then eliminated by metabolism.

### Example 3

Next, the effect of the constitution of the oil core of the NE particles on the premature release of curcumin from the NE particles was examined.

First, the solubility of curcumin in coconut oil or the CAOM produced in Example 1 was examined. The coconut oil was used as a model of triglyceride oils composed of medium chain fatty acids. The solubility of curcumin in coconut oil was very low (2.44±0.24 mg/mL), indicating that it was a poor solvent. The results are shown in FIG. 2. The data represent the mean±standard deviation (SD) (n=3). The solubility of curcumin in CAOM was 6 times higher than the solubility of curcumin in coconut oil, and CAOM significantly improved the solubility of curcumin.

When the solubility of curcumin in purified monoolein dicinnamate and purified monoolein monocinnamate was also measured, there was no clear difference from the solubility of curcumin in CAOM. It was confirmed that the solubility-improving effects of monoolein dicinnamate and monoolein monocinnamate were comparable.

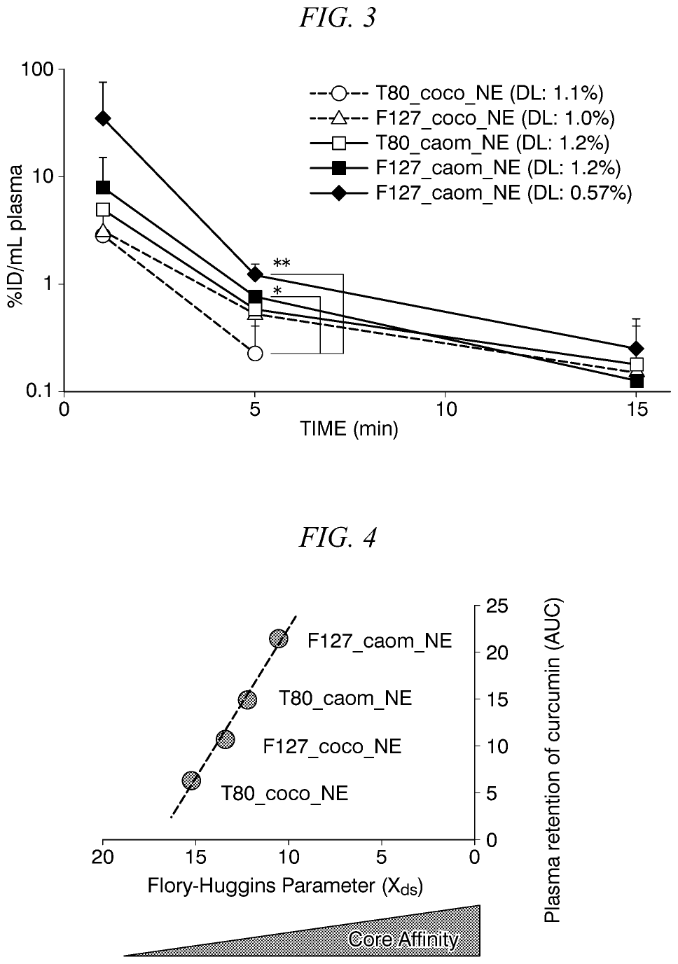

Next, for the four types of NEs listed in Table 1, curcumin-loaded NEs were prepared and injected into ICR mice via the tail vein, and the concentration of curcumin in plasma was measured over time. The measurement results of the relative amount (%) with respect to the initial dose (ID) of curcumin in plasma are shown in FIG. 3. The data represent the mean±SD (n=3). In the figure, “**” indicates that P<0.01 and “*” indicates that P<0.05 (by non-repeated ANOVA followed by Bonferroni correction).

In mice that received T80_coco_NE intravenously, curcumin in plasma was detected only up to 5 minutes after the NE injection. In contrast, in mice that received T80__caom_NE and F127__caom_NE intravenously, curcumin in plasma was detected until 15 minutes after the NE injection. It was speculated that this was due to the formation of it-it stacking between aromatic rings of curcumin and CAOM, which resulted in the long-term retention of curcumin by the oil core of NE.

Further, when the mice that received T80_caom_NE intravenously and the mice that received F127__caom_NE intravenously were compared, the concentration of curcumin in the plasma was higher in the F127__caom_NE injected mice. The same trend was also observed in the comparison between T80_coco_NE and F127_coco_NE. PEG contents of Tween 80 and Pluronic F127 were comparable (67% and 70%, respectively), and there was no difference in the PEGylation density on the NE surface. From this, it is speculated that a hydrogen bond is formed between an etheric oxygen atom in a propylene glycol (PPG) chain, which is a hydrophobic region of Pluronic F127, and a hydroxy group in curcumin, so that the NE using Pluronic F127 may have slowed down the release rate of curcumin in comparison with the NE using Tween 80.

By substituting both the oily component constituting the oil core and the hydrophobic portion of the surfactant with those having a higher affinity against curcumin, it was possible to suppress the premature release of curcumin from the NE particles and to improve the AUC. More specifically, the relative amount of curcumin in plasma 5 minutes after the intravenous injection of NE increased from 0.23% to 0.75% (P<0.05), and the AUC was improved by 3.4 times between the mice intravenously injected with T80 coco_NE and the mice intravenously injected with F127_caom_NE.

FIG. 4 shows a diagram in which Flory-Huggins interaction parameters (X_{ds}) with curcumin in different NE cores (NEs listed in Table 1) are plotted against in vivo stability of curcumin (AUC of plasma profiles). X_{ds} and the AUC of the plasma profiles were in an inversely proportional relationship with a favorable linear fitting (R²=0.985).

From these results, it was found that the AUC can be improved by designing various components constituting the oil core of NE such that the X_{ds} with the material to be solubilized becomes small.

The effects on the toxicity markers of liver function, kidney, and hemolysis in the mice intravenously injected with T80 coco_NE and the mice intravenously injected with F127__caom_NE were investigated 1 hour and 24 hours after intravenous administration. Creatinine (CRE) and blood urea nitrogen (BUN) were examined as plasma markers of kidneys, and alanine transaminase (ALT) and aspartate transaminase (AST) were examined as liver function markers. Lactate dehydrogenase (LDH) was examined as a hemolysis marker. As a result, no sign of toxicity was observed in the mice to which F127__caom_NE was administered intravenously.

### Example 4

The usefulness of CAOM as a dissolving agent of low molecular weight compounds having an aromatic ring other than curcumin was evaluated. Quercetin (Que), which is a polyphenol having an anticancer effect, silicon phthalocyanine dihydroxide (SiPC), which is a compound used in photothermal cancer therapy, and Paclitaxel (PTX), which is well known as a taxane-based anticancer agent were used as 5 the low molecular weight compounds having an aromatic ring.

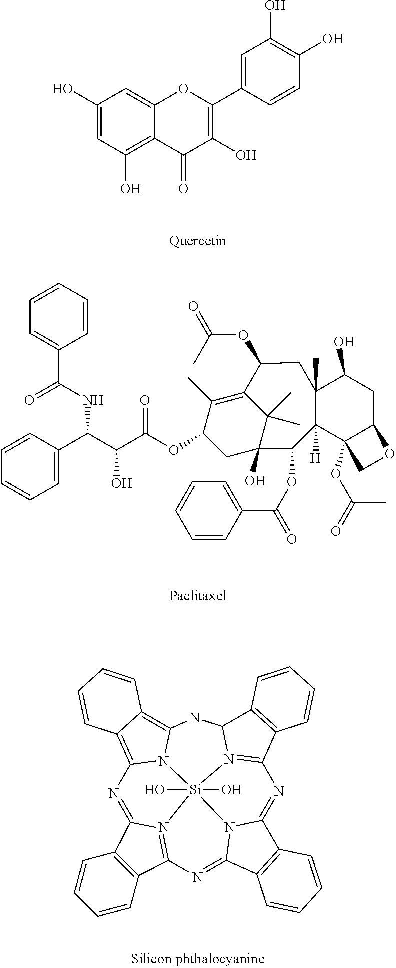

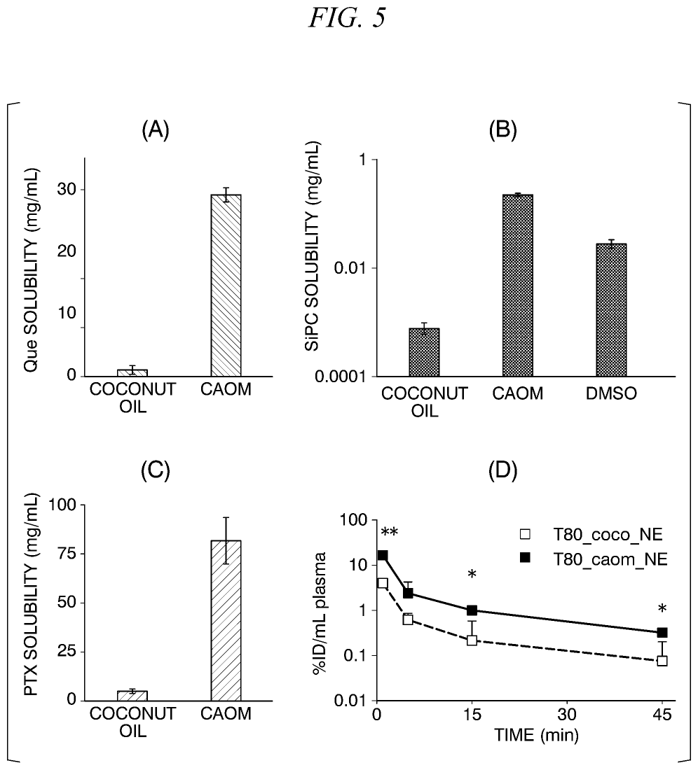

The results of dissolving each compound in CAOM and comparing with coconut oil are shown in FIGS. 5(A) to 5 (C), respectively. The solubility of all of these molecules was significantly improved by CAOM. Further, regarding paclitaxel, the relative amount (%) with respect to the initial dose (ID) of paclitaxel in plasma of the mice intravenously injected with paclitaxel-loaded T80_caom_NE and the mice intravenously injected with paclitaxel-loaded F127__caom_NE was measured in the same manner as in Example 3. The results are shown in FIG. 5(D). The data represent the mean±SD (n=3). In the figure, “**” indicates that P<0.01 and “*” indicates that P <0.05. In the mice intravenously injected with T80_caom_NE, the blood retention of paclitaxel was significantly improved and the AUC was improved by 4.2 times as compared with the mice intravenously injected with T80 coco_NE. The X_{ds} of paclitaxel with the CAOM core was 4.9, while the X_{ds} of paclitaxel with the coconut oil core was 6.8. From these results, it was shown that CAOM improves the solubility of many low molecular weight compounds having an aromatic ring as well as curcumin, and contributes to the improvement of blood retention.

### Example 5

The effect of improving the oral absorption rate of poorly water-soluble drugs by CAOM was examined. As a model drug, fenofibrate (BCS class 2, LogP: 5.3, MW: 361, solubility (in water at 37° C.): <1 μg/mL), which is a therapeutic agent for hyperlipidemia, was used. In addition, seven types of dissolving agents listed in Table 2 were examined. Using each dissolving agent, an NE formulation loaded with fenofibrate was prepared by the method shown in Table 2.

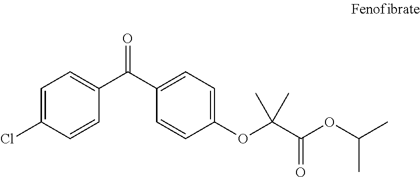 [SMILES: CC(C)OC(=O)C(C)(C)Oc1ccc(C(=O)c2ccc(Cl)cc2)cc1]

TABLE 2

| Name | Constitution/NE preparation method and the like |
|---|---|
| PEG400 | Fenofibrate was dissolved in PEG400 to a concen- |
| | tration of 56 mg/mL. |
| Amorphous solid | Fenofibrate and HPMC were dissolved at a weight |
| dispersion | ratio of 1:2 in t-butyl alcohol, followed by freeze |
| formulation | drying. |
| (ASD) | |
| Corn oil LC | Fenofibrate was dissolved in 32.5% corn oil, 32.5% |
| | Maisine 35-1, and 35% Cremophor EL to a |
| | concentration of 106 mg/mL |
| Labrasol | Fenofibrate was dissolved in 30% Labrasol, 30% |
| | Maisine 35-1, and 40% Cremophor EL to a |
| | concentration of 106 mg/mL |
| CAOM alone | Fenofibrate was dissolved in CAOM to a concen- |
| | tration of 215 mg/mL |
| CAOM LC | Fenofibrate was dissolved in 30% CAOM, 30% |
| | Maisine 35-1, and 40% Cremophor EL to a concen- |
| | tration of 87.3 mg/mL |
| CAOM MC | Fenofibrate was dissolved in 30% CAOM, 30% |
| | Capmul MCM, and 40% Cremophor EL to a concen- |
| | tration of 129 mg/mL |

Each formulation was orally administered to rats so that the amount of fenofibrate was 50 mg/kg, and the amount of fenofibrate in plasma was measured over time. Table 3 shows the plasma AUC and absorption rate (%) of fenofibrate calculated from the measurement result of the amount of fenofibrate. The two formulations containing CAOM (CAOM LC and CAOM MC) showed higher AUC and absorption rate than those of Labrasol, which was an existing formulation. In particular, CAOM LC showed a very high absorption rate of 99%.

TABLE 3

| | AUCₚₗₐₛₘₐ [μg h/ml] | Absorption rate [%] |
|---|---|---|
| PEG400 | 183.30 ± 57.25 | 18 ± 4 |
| ASD | 199.34 ± 9.19 | 20 ± 1 |
| Corn oil LC | 439.48 ± 41.02 | 35 ± 4 |
| Labrasol | 843.06 ± 137.14 | 65 ± 10 |
| CAOM alone | 235.45 ± 203.44 | 24 ± 16 |
| CAOM LC | 951.89 ± 83.31 | 75 ± 6 |
| CAOM MC | 1361.81 ± 124.66 | 99 ± 2 |

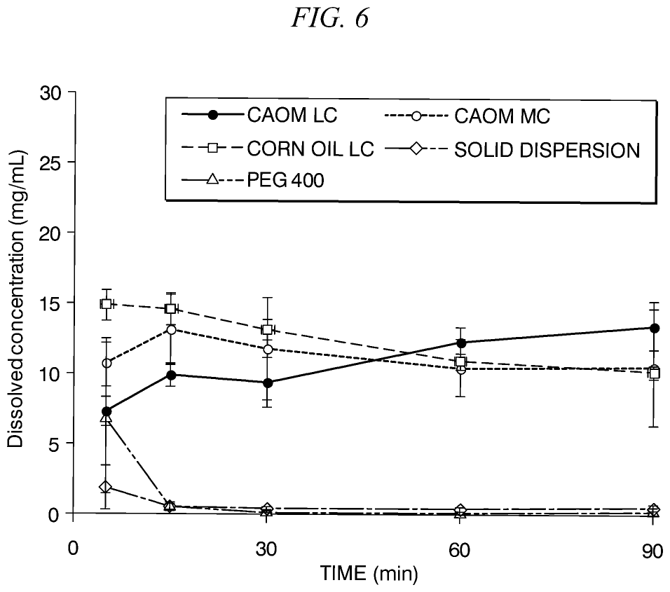

Next, for the purpose of analyzing the absorption improving effects, transition of the in vitro dissolution concentration was examined. Each formulation was added to a model small intestine solution (50 mM sodium taurocholate, 3.7 mM lecithin, pH 6.5) so that the final concentration of fenofibrate was 25 mg/mL, and the concentration of fenofibrate in the solution (mg/mL) up to 90 minutes later was quantified under stirring. The results are shown in FIG. 6. The formulations containing CAOM showed a high fenofibrate concentration of 10 to 15 mg/mL Since the solubility of fenofibrate alone in the model small intestine solution used was 0.0228 mg/mL, it became clear that the formulation containing CAOM significantly increased the apparent solubility of fenofibrate.

Subsequently, in order to verify the safety of the formulation containing CAOM, the damage to the gastrointestinal membrane and the inhibitory effects on P-glycoprotein (P-gp) were examined. First, in order to investigate the damage to the gastrointestinal membrane, CAOM LC and CAOM MC formulations required at the time of oral administration of fenofibrate at 50 mg/kg were orally administered to rats, followed by orally administering low-membrane-permeable drugs (2 mg/mL atenolol and 10 mg/mL FD-4) to determine the plasma AUC of each low-membrane-permeable drug. The results are shown in Table 4. None of the formulations showed a significant change in the plasma AUC of the low-membrane-permeable drug. That is, it was shown that the formulations containing CAOM did not cause membrane damage.

TABLE 4

AUCₚₗₐₛₘₐ [μg h/ml]

| | Atenolol | FD-4 |
|---|---|---|
| Control | 171.90 ± 46.75 | 444.80 ± 32.78 |
| CAOM LC | 191.65 ± 26.80 | 583.39 ± 213.83 |
| CAOM MC | 210.39 ± 36.34 | 495.05 ± 34.60 |

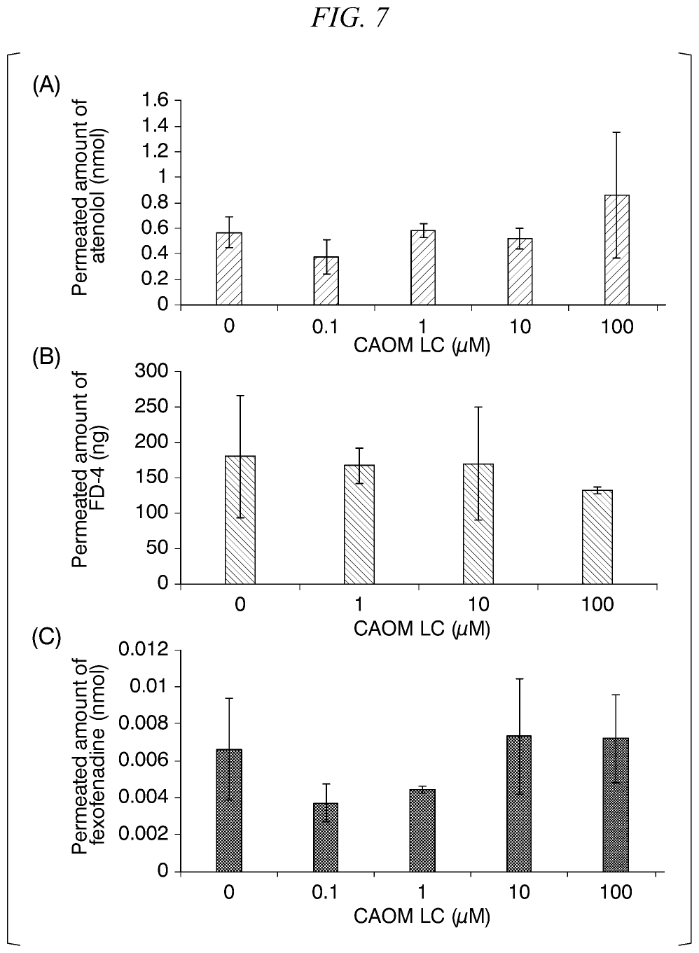

Furthermore, in order to investigate the P-gp inhibitory effect, MDR-MDCKII cells were planarly cultured on transwells, and low-membrane-permeable drugs (1 mM atenolol, 0.1 mg/mL FD-4, and 1 μM fexofenadine) and 0.1 to 100 μM_CAOM_LC were added to the apical side, and the amount of each low-membrane-permeable drug migrated to the basal side was quantified. The results are shown in FIGS. 7(A) to 7(C). As a result, no significant change was observed in the amount of each low-membrane-permeable drug migrated to the basal side by the addition of CAOM LC, indicating that CAOM LC does not exhibit P-gp inhibitory activity.

### Example 6

The solubility of curcumin was investigated in compounds structurally similar to CAOM. As the structurally similar compounds, a mixture of monopalmitin dicinnamate and monopalmitin monocinnamate (monopalmitin cinnamate mixture: Clinn.

Palm), and a mixture of monoolein diphenyl butyrate and monoolein monophenyl butyrate (monoolein phenyl butyrate mixture: PheBut. Ole) were used. These mixtures were synthesized in the same manner as in Example 1. Monopalmitin dicinnamate was the most abundant in the monopalmitin cinnamate mixture, and monopalmitin monocinnamate (1) was more easily synthesized than monopalmitin monocinnamate (2) due to steric hindrance, and the mixture contained more monopalmitin monocinnamate (1) than monopalmitin monocinnamate (2). Similarly, monoolein diphenyl butyrate was the most abundant in the monoolein phenyl butyrate mixture, and monoolein monophenyl butyrate (1) was contained more than monoolein monophenyl butyrate (2).

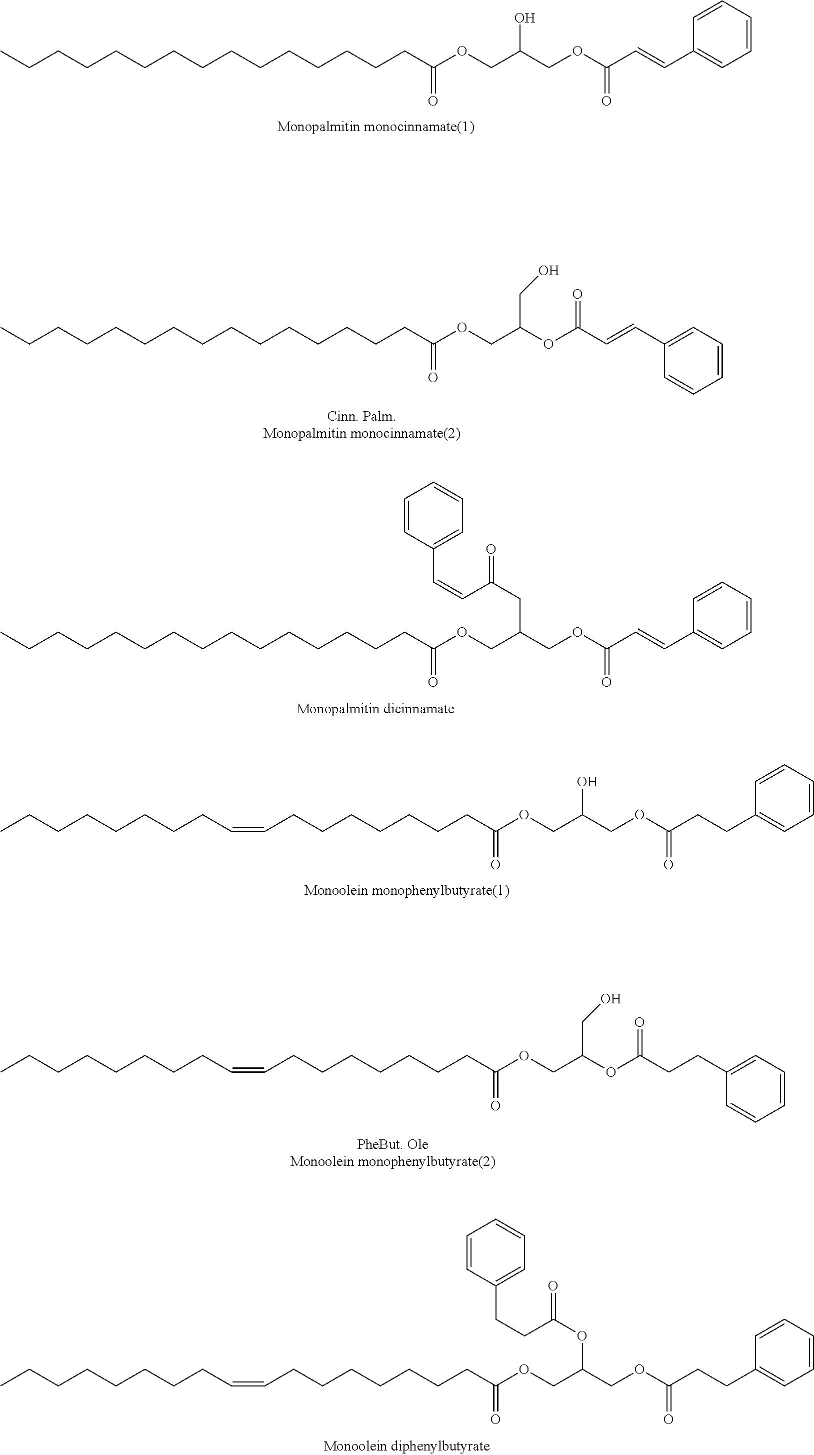 [SMILES: CCCCCCCCCCCCCCCC(=O)OCC(O)COC(=O)/C=C/c1ccccc1 ; CCCCCCCCCCCCCCCC(=O)OCC(CO)OC(=O)/C=C/c1ccccc1 ; CCCCCCCCCCCCCCCC(=O)OCC(COC(=O)/C=C/c1ccccc1)CC(=O)/C=C\c1ccccc1 ; CCCCCCCC/C=C\CCCCCCCC(=O)OCC(O)COC(=O)CCc1ccccc1 ; CCCCCCCC/C=C\CCCCCCCC(=O)OCC(CO)OC(=O)CCc1ccccc1 ; CCCCCCCC/C=C\CCCCCCCC(=O)OCC(COC(=O)CCc1ccccc1)OC(=O)CCc1ccccc1]

Using coconut oil as a control, the solubility of curcumin in the monopalmitin cinnamate mixture and the monoolein phenyl butyrate mixture was examined in the same manner as in Example 3. The solubility of curcumin in the monopalmitin cinnamate mixture was measured at 100° C., which was higher than the melting point of monopalmitin dicinnamate. On the other hand, the solubility of curcumin in the monoolein phenyl butyrate mixture was measured at room temperature in the same manner as the solubility of curcumin in CAOM in Example 3.

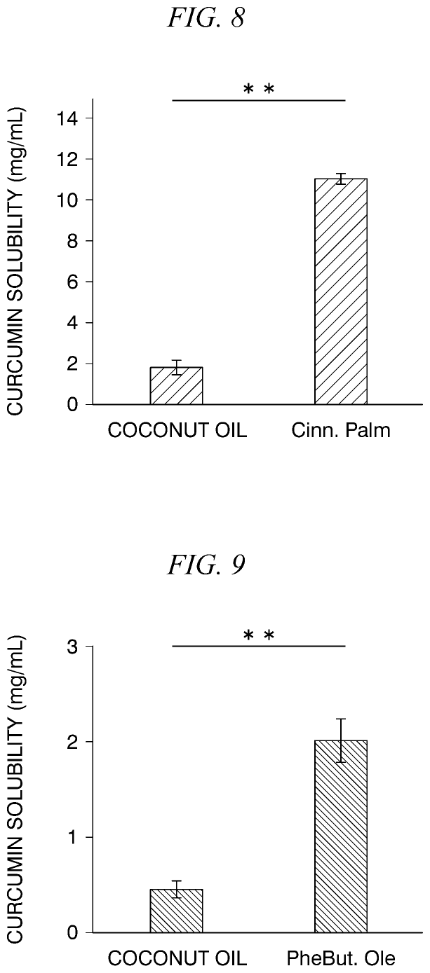

FIG. 8 shows the measurement result of the solubility (mg/mL) of curcumin in the monopalmitin cinnamate mixture, and FIG. 9 shows the measurement result of the solubility (mg/mL) of curcumin in the monoolein phenyl butyrate mixture, respectively. The data represent the mean±SD (n=3). As shown in FIGS. 8 and 9, although slightly inferior to CAOM, both mixtures had much higher curcumin solubilities than that of coconut oil.

1. A compound of general formula (1): 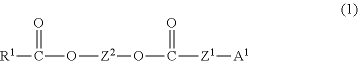
(wherein R¹ is an alkyl group or alkenyl group having 7 to 24 carbon atoms; A¹ is an aryl group which may have a substituent; Z¹ is a single bond, an alkylene group having 1 to 6 carbon atoms, or an alkenylene group having 2 to 6 carbon atoms; and Z² is a divalent linking group).

2. The compound according to claim 1, which is of general formula (2-1) or (2-2): 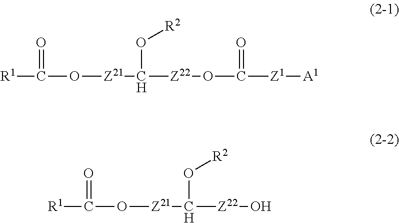
(wherein R¹, Z¹, and A¹ are defined in the formula (1); Z²¹ and Z²² are each independently a single bond, an alkylene group having 1 to 3 carbon atoms, or an alkenylene group having 2 to 3 carbon atoms; R² is a hydrogen atom, —CO—R¹, —NH—R¹, —CO—Z¹-A¹ or —NH—Z¹-A¹, and when a plurality of R¹, Z¹, or A¹ is present, the plurality of R¹, Z¹, or A¹ may be identical to or different from each other).

3. The compound according to claim 1, which is of general formula (3-1), (3-2), or (3-3): 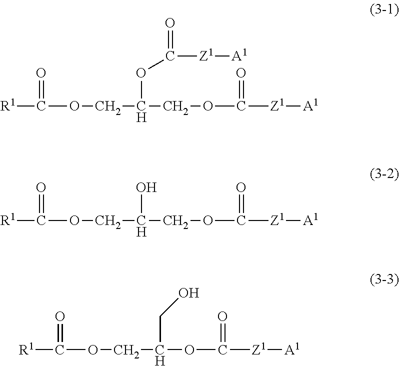
(wherein R¹, Z¹, and A¹ are defined in the formula (1), and in the formula (3-1), two of Z¹ and A¹ may be identical to or different from each other).

4. The compound according to claim 3,
wherein in the general formulae (3-1), (3-2), and (3-3), R¹—COO— is an oleic acid residue or a palmitic acid residue,
Z¹ is a methylene group, an ethylene group or a vinylene group, and
A¹ is a phenyl group which may have a substituent.

5. The compound according to claim 1, which is one or more selected from the group consisting of monoolein dicinnamate, monoolein monocinnamate, monopalmitin dicinnamate, monopalmitin monocinnamate, monoolein diphenyl butyrate, and monoolein monophenyl butyrate.

6. A dissolving agent comprising a compound of claim 1.

7. A method of dissolving a compound having an aromatic ring, comprising using a dissolving agent of claim 6.

8. A pharmaceutical composition comprising a compound of claim 1.

9. The pharmaceutical composition according to claim 8, further comprising a compound having an aromatic ring.
